(19) United States
(12) Patent Application Publication
(10) Pub. No.: US 2026/0130729 A1
(43) Pub. Date: May 14, 2026

(54) INTERFACE FOR TRACKING AND CONTROLLING MOTION, AND APPARATUS AND SYSTEM COMPRISING SAME

(71) Applicant: EWHA UNIVERSITY - INDUSTRY COLLABORATION FOUNDATION, Seoul (KR)

(72) Inventors: Seok Chang RYU, Seoul (KR); Yu Jeong SHIN, Seoul (KR); Se Rin CHA, Seoul (KR); Se Bin KIM, Seoul (KR)

(21) Appl. No.: 19/118,524

(22) PCT Filed: Oct. 4, 2023
(86) PCT No.: PCT/KR2023/015216

§ 371 Date: Apr. 4, 2025

(30) Foreign Application Priority Data
Oct. 7, 2022 (KR) 10-2022-0128705
Aug. 17, 2023 (KR) 10-2023-0107497

(51) Int. Cl. A61B 34/37; A61B 34/20

(52) U.S. Cl. CPC A61B 34/37; A61B 34/20; A61B 2034/2065

(57) ABSTRACT

An embodiment of the present disclosure provides an interface for tracking and controlling a motion, the interface including a base part including a handle part which is holdable by a user, and a main body part including a plurality of links pivotally connected to each other and a finger seating part on which a finger of the user is seated, the main body part being rotatably connected to the base part, wherein at least one of the plurality of links is formed in a shape which is curved in a preset direction.

### TECHNICAL FIELD

The present disclosure relates to an interface for tracking and controlling a motion, and an apparatus and system including the same.

### BACKGROUND ART

In medical terms, surgery refers to curing an illness by cutting, incising, or manipulating the skin, mucous membranes, or other tissues by using medical apparatuses. In particular, open surgery, which involves cutting open the skin at the surgical site and treating, shaping, or removing the organs inside, causes problems such as bleeding, side effects, patient pain, and scarring. Thus, recently, minimal invasive surgery using robots has attracted attention as an alternative.

Minimal invasive surgery has the advantages of less postoperative pain than open surgery, early recovery of bowel movement and early food intake, short hospitalization period, quick return to normal state, and excellent cosmetic effects due to narrow incision range. Due to these advantages, minimal invasive surgery is used in cholecystectomy, prostate cancer surgery, hernia repair, etc., and the applications of minimal invasive surgery are gradually expanding.

Surgical robots commonly used in minimal invasive surgery include a master apparatus and a slave apparatus. The master apparatus generates a control signal according to a manipulation of a medical doctor and transmits the control signal to the slave apparatus. The slave apparatus receives the control signal from the master apparatus and applies manipulations required for surgery to a patient. The master apparatus and the slave apparatus are configured as an integrated apparatus or separate apparatuses and are placed in an operating room to perform surgery.

In this case, research and development is ongoing to minimize the sense of incongruity caused by the kinematic dissimilarity between the master apparatus and the slave apparatus during the process in which the user remotely manipulates the slave apparatus by using the master apparatus.

In the past, a master apparatus has been used to manipulate a slave apparatus by recognizing a movement of a user's two fingers, that is, a user's thumb and index finger, together with a user's wrist. However, in case that it was difficult to implement a wrist joint where the three-degree-of-freedom rotation axes intersect at a single point or at a short distance due to small size, or in case of a slave apparatus without a wrist joint due to surgical necessity, there was a problem in that a user's biomechanical motion characteristics and the slave apparatus are not similar kinematically, and thus, a non-intuitive problem occurring while the user manipulated the slave apparatus could not be solved.

### DISCLOSURE OF INVENTION

### Technical Problem

The present disclosure provides an interface for tracking and controlling a motion, which is capable of precisely and intuitively controlling a slave robot corresponding kinematically thereto by detecting rotation angles of a plurality of links and joint parts which operate according to the motion of the finger of the user.

### Solution to Problem

An aspect of the present disclosure provides an interface for tracking and controlling a motion, the interface including a base part including a handle part which is holdable by a user, and a main body part including a plurality of links pivotally connected to each other and a finger seating part on which a finger of the user is seated, the main body part being rotatably connected to the base part, wherein at least one of the plurality of links is formed in a shape which is curved in a preset direction.

### Advantageous Effects of Invention

Because an interface for tracking and controlling a motion, according to an embodiment of the present disclosure, includes a plurality of links pivotally connected to each other and a finger seating part on which a finger of a user is seated, there is an effect that may effectively detect the degree of bending of the finger and may intuitively control a robot which is kinematically similar thereto.

In addition, because a base part is rotatably connected to a manipulator, there is an effect that may additionally detect the degree of adduction/abduction of a wrist of the user.

### BRIEF DESCRIPTION OF DRAWINGS

FIG. 1 is a diagram schematically illustrating a surgical robot system according to an embodiment of the present disclosure.

FIG. 2 is a diagram for describing a motion of an end effector according to an embodiment of the present disclosure.

FIG. 3 is a perspective view of a master apparatus according to an embodiment of the present disclosure.

FIG. 4 is a perspective view of an interface for tracking and controlling a motion, according to an embodiment of the present disclosure.

FIG. 5 is a side view of a main body part according to an embodiment of the present disclosure.

FIG. 6 is an exploded view of the main body part according to an embodiment of the present disclosure.

FIGS. 7 and 8 are diagrams for describing a usage state of an interface for tracking and controlling a motion, according to an embodiment of the present disclosure.

FIG. 9 is a perspective view of a master apparatus according to another embodiment of the present disclosure.

FIG. 10 is a perspective view of a laparoscopic surgical apparatus according to an embodiment of the present disclosure.

### BEST MODE FOR CARRYING OUT THE INVENTION

An aspect of the present disclosure provides an interface for tracking and controlling a motion, the interface including a base part including a handle part which is holdable by a user, and a main body part including a plurality of links pivotally connected to each other and a finger seating part on which a finger of the user is seated, the main body part being rotatably connected to the base part, wherein at least one of the plurality of links is formed in a shape which is curved in a preset direction.

In addition, the plurality of links may include a first link connected to the base part so as to be rotatable about a first rotation axis, a second link connected to the first link so as to be rotatable about a second rotation axis, and a third link connected to the second link so as to be rotatable about a third rotation axis and connected to the finger seating part so as to be rotatable about a fourth rotation axis.

In addition, the second rotation axis, the third rotation axis, and the fourth rotation axis may be parallel to each other.

In addition, the first rotation axis and the second rotation axis may cross each other

In addition, the first rotation axis and the second rotation axis may be arranged perpendicular to each other.

In addition, an angle formed by the second rotation axis and a longitudinal central axis of the handle part may be 30°or less.

In addition, the first rotation axis and a longitudinal central axis of the handle part may cross each other.

In addition, the first rotation axis and the longitudinal central axis of the handle part may be arranged perpendicular to each other.

In addition, the main body part may include a first joint part which rotatably connects the base part to the first link, a second joint part which rotatably connects the first link to the second link, a third joint part which rotatably connects the second link to the third link, and a fourth joint part which rotatably connects the third link to the finger seating part, wherein at least one of the first joint part, the second joint part, the third joint part, and the fourth joint part may include a sensor part capable of detecting a change in rotation angles of the plurality of links.

In addition, the sensor part may include either a magnetic rotary encoder or an optical rotary encoder.

In addition, a sensor part connected to the first joint part and a sensor part connected to the second joint part may include different types of encoders.

In addition, at least one of the first joint part, the second joint part, the third joint part, and the fourth joint part may further include a load providing part configured to provide preset torque between links connected to each other.

In addition, the handle part may include a manipulation part configured to selectively generate a preset control command according to a manipulation of a user.

In addition, the finger seating part may include a haptic module capable of transmitting external environment information to a user.

Another aspect of the present disclosure provides a master apparatus including a manipulator including a plurality of robot joints which are rotatable about one or more non-parallel rotation axes, and an interface for tracking and controlling a motion which is coupled to an end portion of the manipulator and is capable of tracking a hand motion of a user, wherein the interface for tracking and controlling a motion includes a base part including a handle part capable of coming into contact with a palm of the user and a fastening part coupled to the end portion of the manipulator, and a main body part including a plurality of links pivotally connected to each other and a finger seating part on which a finger of the user is seated, the main body part being rotatably connected to the base part.

In addition, the plurality of links may include a first link connected to the base part so as to be rotatable about a first rotation axis, a second link connected to the first link so as to be rotatable about a second rotation axis, and a third link connected to the second link so as to be rotatable about a third rotation axis and connected to the finger seating part so as to be rotatable about a fourth rotation axis.

In addition, the interface for tracking and controlling a motion may be rotatable about a preset axis passing through the fastening part, and the preset axis may be spaced apart from the first rotation axis.

In addition, the fastening part may be detachable from the handle part.

Another aspect of the present disclosure provides a laparoscopic surgical apparatus including a surgical instrument including an end effector for surgery and a shaft configured to transmit a driving force for a motion of the end effector, and an interface for tracking and controlling a motion which is coupled to an end portion of the surgical instrument and is configured to track a hand motion of a user according to a manipulation and transmit hand motion information to the end effector, wherein the interface for tracking and controlling a motion includes a base part including a handle part capable of coming into contact with a palm of the user and a fastening part coupled to the end portion of the surgical instrument, and a main body part including a plurality of links pivotally connected to each other and a finger seating part on which a finger of the user is seated, the main body part being rotatably connected to the base part.

Another aspect of the present disclosure provides a surgical robot system including a master apparatus including a manipulator including a plurality of robot joints which are rotatable about one or more non-parallel rotation axes and an interface for tracking and controlling a motion which is coupled to an end portion of the manipulator and is capable of tracking a hand motion of a user, and a slave robot which is driven by receiving an electric signal from the master apparatus according to a movement of the master apparatus, wherein the interface for tracking and controlling a motion includes a base part including a handle part which is holdable by the user, and a main body part including a plurality of links pivotally connected to each other and a finger seating part on which a finger of the user is seated.

### Mode for the Invention

As the present description allows for various changes and numerous embodiments, certain embodiments will be illustrated in the drawings and described in detail in the written description. Effects and features of the present disclosure, and methods of achieving them will be clarified with reference to embodiments described below in detail with reference to the drawings. However, the present disclosure is not limited to the following embodiments and may be embodied in various forms.

Hereinafter, embodiments will be described in detail with reference to the accompanying drawings. When describing embodiments with reference to the accompanying drawings, the same or corresponding elements are denoted by the same reference numerals, and redundant descriptions thereof are omitted.

The singular forms as used herein are intended to include the plural forms as well unless the context clearly indicates otherwise.

It will be further understood that the terms “include” and/or “comprise” used herein specify the presence of stated features or elements, but do not preclude the presence or addition of one or more other features or elements.

When a certain embodiment is implemented differently, a specific process sequence may be performed differently from a sequence described herein. For example, two consecutively described processes may be performed substantially at the same time or performed in an order opposite to the stated order.

Also, sizes of elements in the drawings may be exaggerated or reduced for convenience of explanation. For example, since sizes and thicknesses of elements in the drawings are arbitrarily illustrated for convenience of explanation, the following embodiments are not limited thereto.

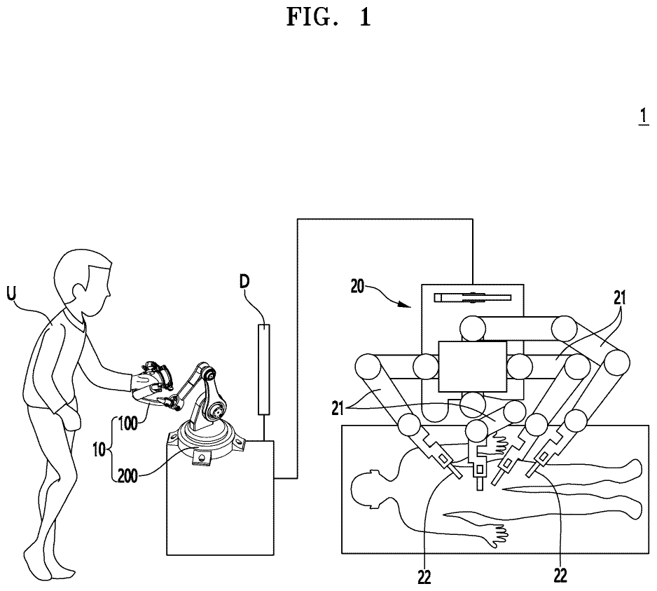

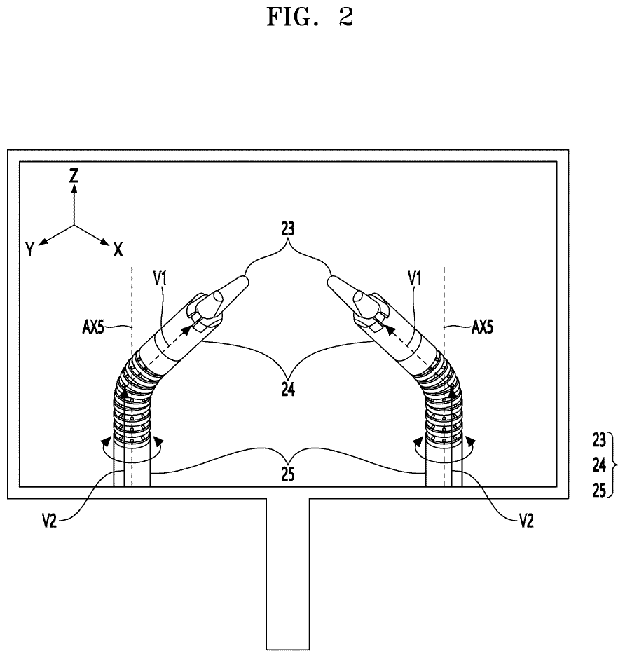

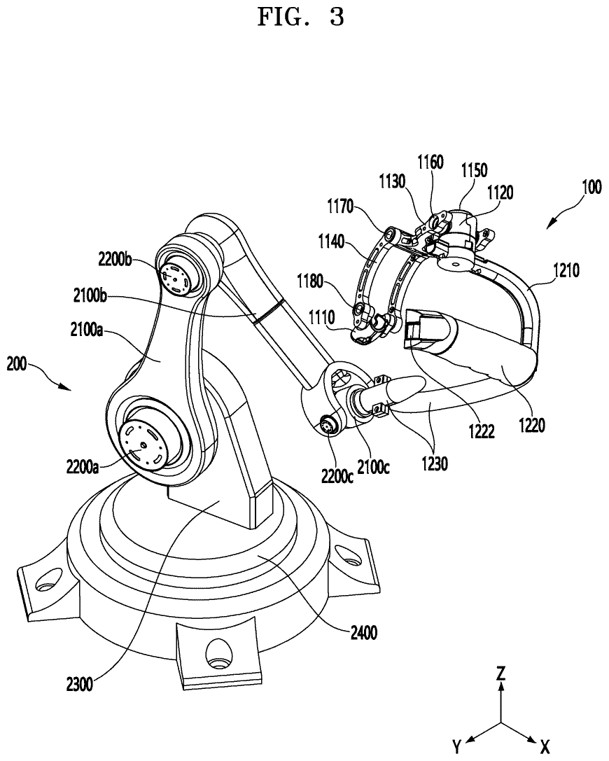

FIG. 1 is a diagram schematically illustrating a surgical robot system according to an embodiment of the present disclosure, FIG. 2 is a diagram for describing a motion of an end effector according to an embodiment of the present disclosure, and FIG. 3 is a perspective view of a master apparatus according to an embodiment of the present disclosure.

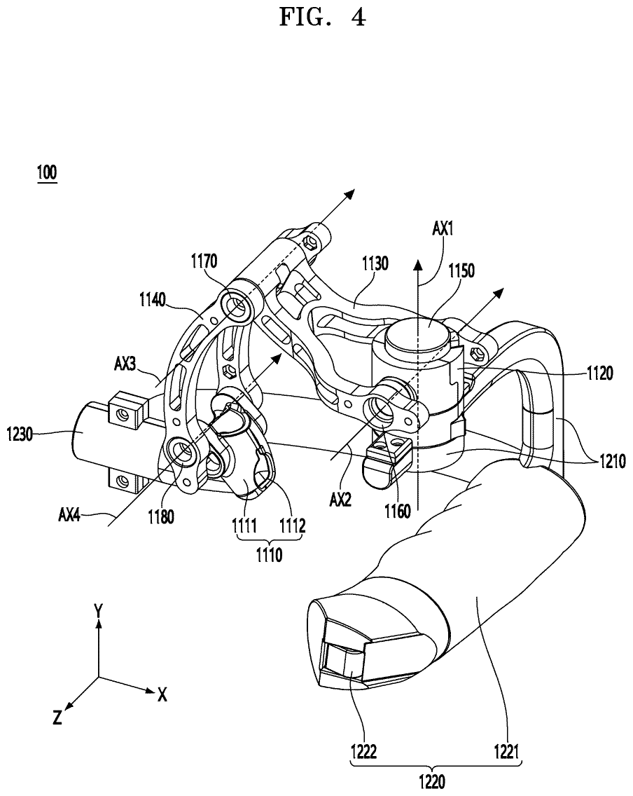

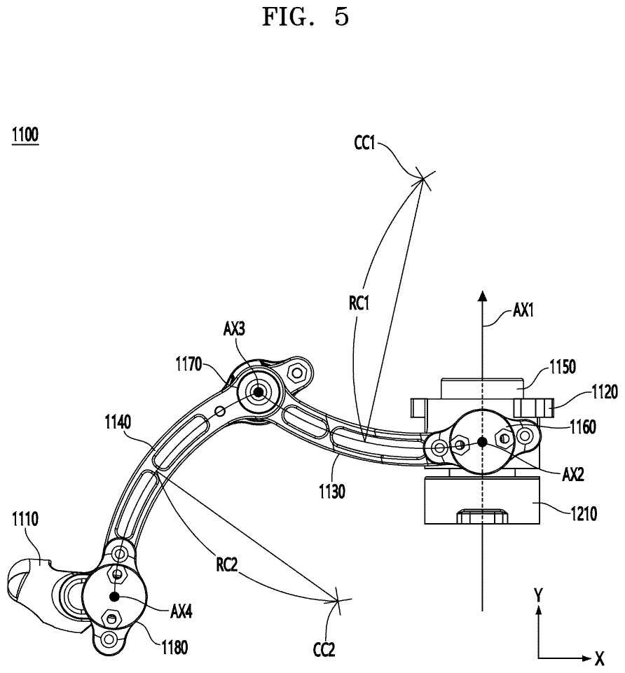

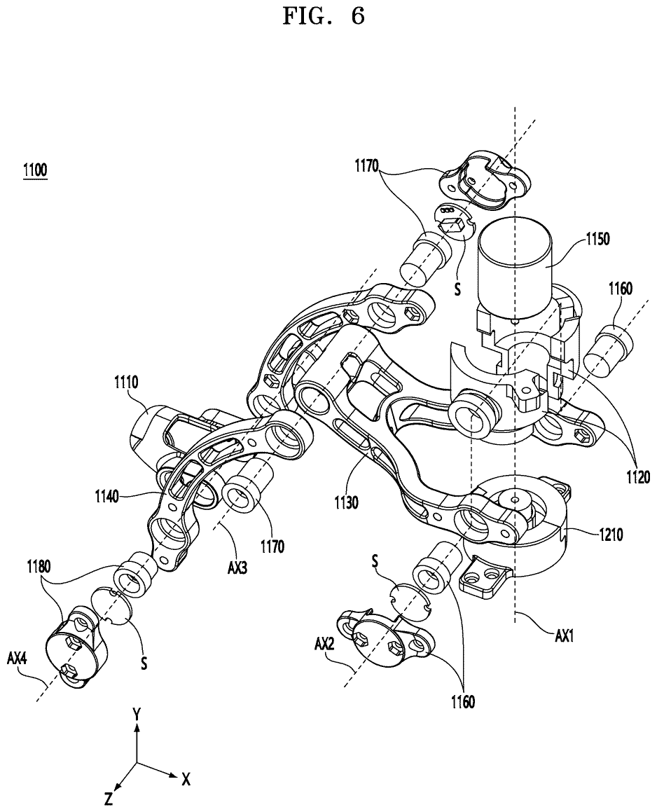

FIG. 4 is a perspective view of an interface for tracking and controlling a motion, according to an embodiment of the present disclosure, FIG. 5 is a side view of a main body part according to an embodiment of the present disclosure, and FIG. 6 is an exploded view of the main body part according to an embodiment of the present disclosure.

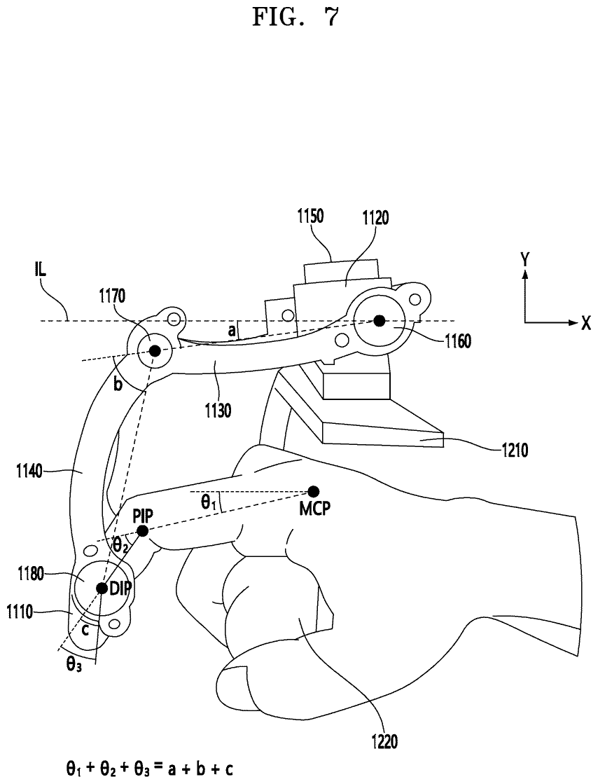

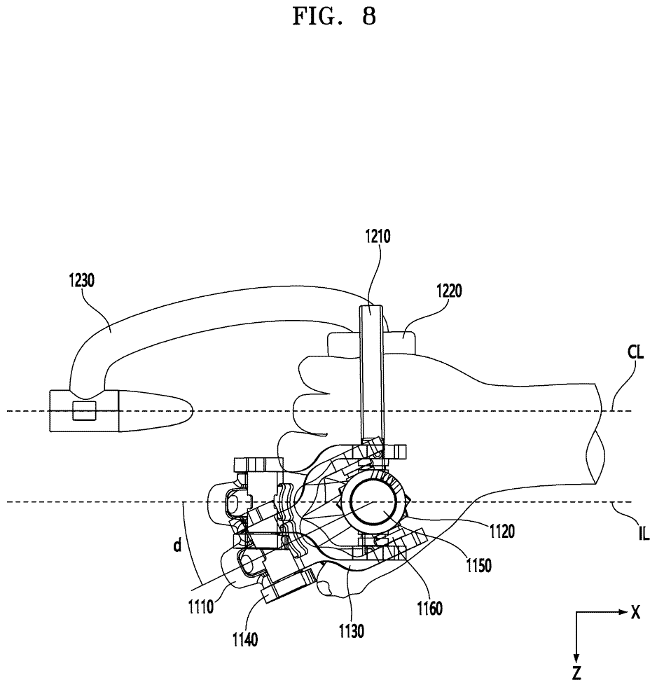

FIGS. 7 and 8 are diagrams for describing a usage state of an interface for tracking and controlling a motion, according to an embodiment of the present disclosure,

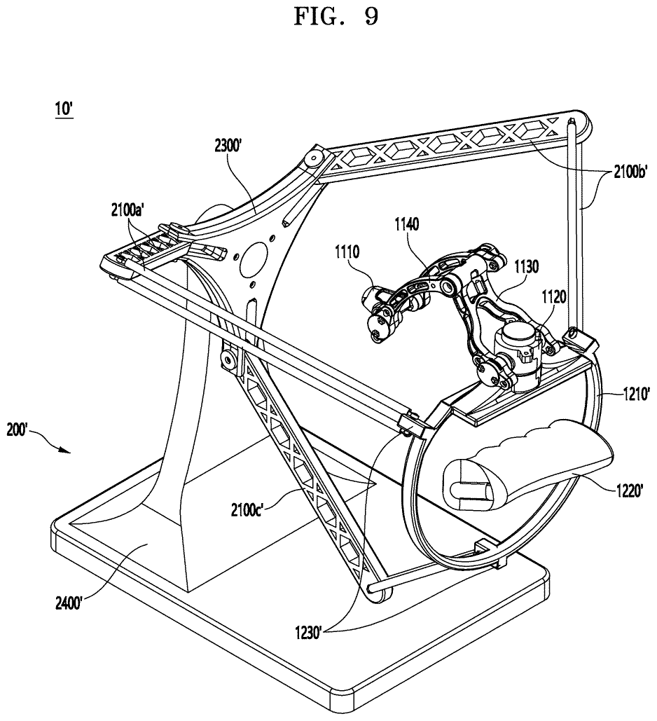

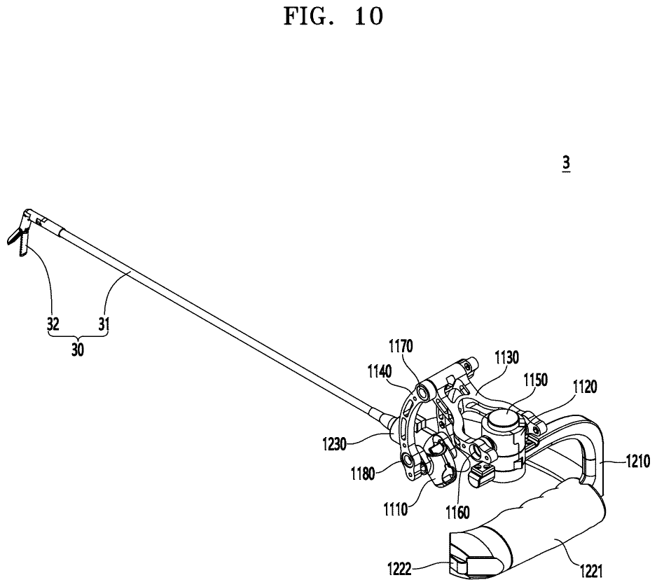

FIG. 9 is a perspective view of a master apparatus according to another embodiment of the present disclosure, and FIG. 10 is a perspective view of a laparoscopic surgical apparatus according to an embodiment of the present disclosure.

A surgical robot system 1 according to an embodiment of the present disclosure is a system which allows a user U to perform surgery remotely by manipulating a slave robot 20 at a remote location using a master apparatus 10.

The master apparatus 10 is electrically connected to the slave robot 20, and the user U who performs surgery may indirectly perform surgery while watching a surgical situation through a display part D and manipulating the master apparatus 10.

An interface 100 for tracking and controlling a motion and the master apparatus 10 including the same, according to an embodiment of the present disclosure, may measure a position of a hand, a rotation angle of a wrist, and a degree of bending of a finger by reflecting ergonomic characteristics. This may provide a high level of intuitiveness to the user U when manipulating the interface 100 for tracking and controlling a motion.

In addition, the shape of the interface 100 for tracking and controlling a motion is formed to be kinematically similar to the shape of the end effector 22 which is connected to an end portion of the slave robot 20 and directly performs surgery. Accordingly, the user U may have high visual intuitiveness when manipulating the interface 100 for tracking and controlling a motion while watching an image captured by the end effector 22.

Hereinafter, the interface 100 for tracking and controlling a motion, as described above, and the apparatuses 3, 10, and 10′and the system 1 including the same are described in detail.

Referring to FIG. 1, the surgical robot system 1 may include the master apparatus 10, the slave robot 20, and the display part D.

The user U who performs surgery may observe a surgical site and the end effector 22, which may come into contact with the surgical site, through the display part, and may perform precise surgery by manipulating the master apparatus 10, specifically the interface 100 for tracking and controlling a motion, to control the motion of the end effector 22 positioned at a remote location.

Referring to FIGS. 1 and 2, the master apparatus 10 according to an embodiment of the present disclosure may include the interface 100 for tracking and controlling a motion and a manipulator 200.

In an embodiment, the interface 100 for tracking and controlling a motion, which is held by a hand of the user U, may be coupled to an end portion of the manipulator 200 which includes a plurality of robot joints 2200a, 2200b, and 2200c rotatable about one or more non-parallel rotation axes.

In an embodiment, the position of the interface 100 for tracking and controlling a motion, which is coupled to the end portion of the manipulator 200, may change as the hand of the user U moves.

In case that the user U holds and operates the interface 100 for tracking and controlling a motion, each of the robot joints 2200a, 2200b, and 2200c of the manipulator 200 is rotatable according to the motion of the user U, and a position value of the manipulator 200 may be calculated by using a rotation angle of each of the robot joints 2200a, 2200b, and 2200c.

Specifically, the robot joints 2200a, 2200b, and 2200c of the manipulator 200 are rotated as the position of the hand of the user U holding and moving the interface 100 for tracking and controlling a motion is changed, and the master apparatus 10 may control the position of the end effector 22 by using the rotation angles of the robot joints 2200a, 2200b, and 2200c.

In an embodiment, the end portion of the manipulator 200 may move with seven-degrees of freedom (DOF), and a vector value V2 of a rear end portion 25 of the end effector 22 (hereinafter, the ‘vector value of the rear end portion 25’ means a vector value of a longitudinal central axis of the rear end portion 25 in an absolute coordinate system) may be determined by using the position and posture of the end portion of the manipulator 200 corresponding to the hand motion of the user U.

In an embodiment, the vector value V2 of the rear end portion 25 may determine the overall position and posture of the end effector 22, and the master apparatus 10 may control the vector value V2 of the rear end portion 25 by using the position and posture of the end portion of the manipulator 200, which change as the hand of the user U holds and rotates the interface 100 for tracking and controlling a motion.

In an embodiment, the interface 100 for tracking and controlling a motion may be rotatably connected to the end portion of the manipulator 200, and the interface 100 for tracking and controlling a motion may be rotated about a central axis CL as the wrist of the user U is abducted/adducted.

In an embodiment, the central axis CL may be parallel to a longitudinal central axis of an upper arm of the user U holding the interface 100 for tracking and controlling a motion.

In an embodiment, the central axis CL may be arranged to cross a longitudinal central axis of a handle part 1220.

In an embodiment, an angle formed by the central axis CL and the longitudinal central axis of the handle part 1220 may be formed to be 30°or less.

In an embodiment, the central axis CL and the longitudinal central axis of the handle part 1220 may be perpendicular to each other.

In case that the wrist of the user U holding the handle part 1220 is abducted/adducted, the interface 100 for tracking and controlling a motion and the end portion of the manipulator 200 are rotated about the central axis CL and the master apparatus 10 may rotate the end effector 22 about a fifth rotation axis AX5 by using the rotation angle of the interface 100 for tracking and controlling a motion, which is rotated about the central axis CL.

For example, in case the user U holds the handle part 1220 and rotates the wrist by α°, the interface 100 for tracking and controlling a motion is rotated about the central axis CL by α°, and the slave robot 20 which has received rotation information of the interface 100 for tracking and controlling a motion may rotate the rear end portion 25 about the fifth rotation axis AX5 by α°.

Accordingly, in case that the user U holds the handle part 1220 and adducts or abducts the wrist, the rear end portion 25 of the end effector 22 corresponding to the wrist of the user U is rotated in a clockwise or counterclockwise direction, and thus, the intuitiveness of the manipulation of the user U may be improved due to the kinematic similarity between the interface 100 for tracking and controlling a motion and the end effector 22.

In an embodiment, the longitudinal central axis of the handle part 1220 may be arranged on the same plane as the central axis CL.

In an embodiment, the user U may hold the handle part 1220 with his/her middle, ring, and index fingers, and the central axis CL may pass through an area of the handle part 1220 where the ring finger of the user U is positioned.

In an embodiment, a fastening part 1230 and the area of the handle part 1220 where the ring finger of the user U is positioned may both be positioned on the central axis CL.

In the present disclosure, the rotation central axis CL of the interface 100 for tracking and controlling a motion is aligned with the axis passing through the ring finger by using the biomechanical feature that the abduction/adduction rotation central axes of the upper arm and the wrist pass through the ring finger. This minimizes the sense of incongruity in manipulation while the user U holds the handle part 1220 and adducts/abducts and also minimizes fatigue which occurs during long-term manipulation. Referring to FIGS. 4 to 6, the interface 100 for tracking and controlling a motion, according to an embodiment of the present disclosure, may include a main body part 1100, which is coupled to the end portion of the manipulator 200 and is capable of tracking the hand motion of the user U, and a base part 1200.

Referring to FIGS. 5 and 6, the main body part 1100 according to an embodiment of the present disclosure may include a plurality of links 1120, 1130, and 1140 pivotally connected to each other and a finger seating part 1110 on which the finger of the user U is seated.

In an embodiment, the plurality of links 1120, 1130, and 1140 may include a first link 1120 connected to the base part 1200 so as to be rotatable about a first rotation axis AX1, a second link 1130 connected to the first link 1120 so as to be rotatable about a second rotation axis AX2, and a third link 1140 connected to the second link 1130 so as to be rotatable about a third rotation axis AX3 and connected to the finger seating part 1110 so as to be rotatable about a fourth rotation axis AX4.

In an embodiment, the first link 1120, the second link 1130, the third link 1140, and the finger seating part 1110 may be connected in series to each other.

In an embodiment, the main body part 1100 may include a first joint part 1150 which rotatably connects the base part 1200 to the first link 1120, a second joint part 1160 which rotatably connects the first link 1120 to the second link 1130, a third joint part 1170 which rotatably connects the second link 1130 to the third link 1140, and a fourth joint part 1180 which rotatably connects the third link 1140 to the finger seating part 1110.

In the present specification, a ‘joint’ refers to a connection point between the links and may have one or more DOF. Here, the ‘DOF’ refers to the degree of freedom in kinematics or inverse kinematics. The DOF of a mechanism refers to the number of independent motions of the mechanism, or the number of variables which determine independent motions of relative positions between the respective links. For example, an object in a three-dimensional space consisting of an x-axis, a y-axis, and a z-axis has at least one of three-DOF (a position at each axis) for determining the spatial position of the object, three-DOF (a position at each axis) for determining the spatial posture of the object, and three-DOF (a rotation angle for each axis) for determining the spatial posture of the object.

In an embodiment, at least one of the first joint part 1150, the second joint part 1160, the third joint part 1170, and the fourth joint part 1180 may be formed in various shapes capable of a one-DOF rolling motion.

In an embodiment, the first joint part 1150 may include a revolute joint which connects the base part 1200 to the first link 1120 so as to be rotatable about the first rotation axis AX1.

In an embodiment, the first joint part 1150 may be formed in a shape of a bearing which connects the base part 1200 to the first link 1120 so as to be rotatable about the first rotation axis AX1.

In an embodiment, the second joint part 1160 may include a revolute joint which connects the first link 1120 to the second link 1130 so as to be rotatable about the second rotation axis AX2.

In an embodiment, the third joint part 1170 may include a revolute joint which connects the second link 1130 to the third link 1140 so as to be rotatable about the second rotation axis AX2.

In an embodiment, the fourth joint part 1180 may include a revolute joint which connects the second link 1130 to the third link 1140 so as to be rotatable about the second rotation axis AX2.

In an alternative embodiment, at least one of the first to fourth joint parts 1150, 1160, 1170, and 1180 may be formed as one or more of a cylindrical joint, a universal joint, or a spherical joint, and may be provided with various connection structures capable of connecting the finger seating part 1110 to the adjacent links 1120, 1130, and 1140 so as to be rotatable about a preset axis.

Due to the physical characteristics, a metacarpophalangeal joint (MCP) is capable of both abduction/adduction and flexion (MP joint flexion)/extension (MP joint extension). Accordingly, each of the first joint part 1150 and the second joint part 1160 may be connected to the first link 1120, the first joint part 1150 may be rotated about the first rotation axis AX1 parallel to the abduction/adduction axis of the MCP joint, and the second joint part 1160 may be rotated about the second rotation axis AX2 parallel to the rotation axis of flexion/extension of the MCP joint.

Referring to FIG. 6, at least one of the first joint part 1150, the second joint part 1160, the third joint part 1170, and the fourth joint part 1180 according to an embodiment of the present disclosure may include a sensor part S capable of detecting force/torque information applied to the joint, position information of the joint, moving speed information, and the like.

In an embodiment, the sensor part S may include either a magnetic rotary encoder or an optical rotary encoder.

Although not illustrated in the drawings, the master apparatus 10 according to an embodiment of the present disclosure may include an imaging part capable of capturing motions of the finger seating part 1110, the plurality of links 1120, 1130, and 1140, the joint parts 1150, 1160, 1170, and 1180, and the base part 1200, and the imaging part may detect the position and the rotation angle of at least one of the finger seating part 1110, the plurality of links 1120, 1130, and 1140, the joint parts 1150, 1160, 1170, and 1180, and the base part 1200.

In an embodiment, the imaging part may include an RGB camera. In this case, the imaging part may detect a wavelength of a red, green, or blue band and may generate digital code (RGB) corresponding to the wavelength of each band. However, the present disclosure is not limited thereto, and the imaging part may detect wavelengths of various bands and may generate a digital code preset to correspond to the wavelength of each band.

In addition, the imaging part may amplify an electric signal and output image information based on the amplified electric signal to a control unit which is separately provided. This has an effect of obtaining an image of the interface 100 for tracking and controlling a motion even in an external environment with low illuminance.

In an embodiment, the imaging part may be any device capable of capturing a three-dimensional image of an environment including a human body and obtaining depth information for each pixel of the captured image. For example, the imaging part may include a device capable of calculating a distance value of an object included in a capturing area, such as an infrared radiation camera, a stereo camera, a time-of-flight (TOF) camera, or a kinect depth camera.

For example, in case that the imaging part includes an infrared radiation camera, the imaging part may project infrared light and may generate a human body image of a target human body by using a phase value difference between the projected infrared light and the received infrared light. A cycle in which the imaging part projects infrared light may be continuous or discontinuous. The cycle in which the imaging part projects infrared light may be preset.

In another embodiment, the imaging part may include an RGBD camera that includes an RGB camera which obtains an RGB image by capturing a direction in which a human body is located, and a depth camera which obtains distance information between the user, the imaging part, and any point on the human body.

In this case, the imaging part may determine coordinates for the position of the human body on coordinates for the entire area of the RGB image by mapping the distance information obtained through the depth camera to the RGB image obtained through the RGB camera (in computer graphics, mapping refers to a task of performing conversion from one coordinate system to another), and the imaging part may generate the human body image through the RGB image to which depth information is mapped.

The imaging part may capture the motions of the main body part 1100 and the base part 1200 at a preset frame rate and may generate an image of the main body part 1100 and the base part 1200 captured at a speed corresponding to the frame rate. Due to this, the control unit which is separately provided may detect frame-based motion information of the interface for tracking and controlling a motion, which is generated by the imaging part, in units of frames.

Accordingly, the imaging part may track the motions of the finger seating part 1110, the plurality of links, the different joint parts 1150, 1160, 1170, and 1180, and the base part 1200 by capturing an image of the interface for tracking and controlling a motion and detecting the behaviors of the main body part 1100 and the base part 1200.

In an embodiment, a plurality of optical markers may be attached to the interface for tracking and controlling a motion. Specifically, the plurality of optical markers may be attached to a preset position of at least one of the finger seating part 1110, the plurality of links 1120, 1130, and 1140, the different joint parts 1150, 1160, 1170, and 1180, and the base part 1200.

In this case, the imaging part may include a tracking camera capable of detecting geometric information of the marker, such as the position, the rotation speed, etc. of the optical marker.

Accordingly, the imaging part may track the motions of the finger seating part 1110, the plurality of links 1120, 1130, and 1140, the different joint parts 1150, 1160, 1170, and 1180, and the base part 1200 by capturing an image of the marker. In an embodiment, the sensor part S connected to the first joint part 1150 and the sensor part S connected to the second joint part 1160 may include different types of encoders. Specifically, the sensor part S connected to the first joint part 1150 may include an optical encoder, and the sensor part S connected to the second joint part 1160 may include a magnetic encoder.

Accordingly, in case that the first joint part 1150 and the second joint part 1160 are connected to the first link 1120 and arranged adjacent to each other so as to be rotatable about the axes crossing each other, a magnetic field interference effect between the magnetic encoders attached to the respective joint parts may be prevented.

Although not illustrated in the drawings, at least one of the first joint part 1150, the second joint part 1160, the third joint part 1170, and the fourth joint part 1180 according to an embodiment of the present disclosure may further include a load providing part (not shown) which provides preset torque between the links connected to each other.

The load providing part provides an opposing rotational moment to at least one of the first link 1120, the second link 1130, the third link 1140, and the finger seating part 1110, and may include a torsion spring, a driving motor, etc.

However, the present disclosure is not limited thereto, and the load providing part may be formed in various structures within the technical concept which is capable of providing a moment in one direction (in a clockwise direction in FIG. 5) to each joint part, such as an elastic member which connects the adjacent links and the finger seating part 1110, 1120, 1130, and 1140 or the adjacent joint parts 1150, 1160, 1170, and 1180.

In an embodiment, the load providing part may provide a rotational moment in a clockwise direction (in FIG. 5).

Accordingly, even when the user U holds the finger seating part 1110 with his/her finger and then performs a finger bending motion, the load providing part provides a moment opposite to a finger bending moment at each joint, thereby preventing a phenomenon that the finger is released from the finger seating part 1110.

Referring to FIG. 8, the first link 1120 may be rotatably connected to a base frame 1210 which connects the handle part 1220 to the main body part 1100 through the first joint part 1150.

In an embodiment, the first link 1120 may be formed in a shape of a circular tube extending along the first rotation axis AX1. However, the present disclosure is not limited thereto, and the first link 1120 may be formed in various shapes that are connected to the base frame 1210 so as to be rotatable about the first rotation axis AX1.

In an embodiment, in case that the rotation central axis of the interface 100 for tracking and controlling a motion is parallel to a first direction (an X-axis direction in FIG. 4), the handle part 1220 may be formed to extend in a second direction (a Z-axis direction in FIG. 4) and the first link 1120 may extend in a third direction (a Y-axis direction in FIG. 4).

In an embodiment, the first rotation axis AX1 may cross the longitudinal central axis of the fastening part 1230.

In an embodiment, the first rotation axis AX1 may be perpendicular to the longitudinal central axis of the fastening part 1230.

In an embodiment, the first rotation axis AX1 may cross the longitudinal central axis of the handle part 1220.

In an embodiment, the first rotation axis AX1 may be perpendicular to the longitudinal central axis of the handle part 1220.

In an embodiment, the first rotation axis AX1 may be perpendicular to a plane including the fastening part 1230 and the handle part 1220.

In an embodiment, in case that the rotation central axis of the handle part 1220 is parallel to the X-axis (in FIG. 4), the first rotation axis AX1 may be arranged parallel to the Y-axis (in FIG. 4).

In an embodiment, the first rotation axis AX1 may overlap an area of the handle part 1220 where the index finger is positioned.

In an embodiment, the first rotation axis AX1 may coincide with the rotation axis of abduction/adduction of the MCP of the hand holding the handle part 1220.

In an embodiment, the first rotation axis AX1 may overlap an area of the handle part 1220 where the MCP is positioned.

In an embodiment, the first link 1120 may be connected to the finger seating part 1110 through the second link 1130 and the third link 1140. As the finger seating part 1110 is rotated about the first rotation axis AX1, the first link 1120 may be rotated about the first rotation axis AX1 in a relationship with the base frame 1210.

Each of the second rotation axis AX2, the third rotation axis AX3, and the fourth rotation axis AX4 is not parallel to the first rotation axis AX1. Accordingly, an angle at which the finger seating part 1110 is rotated about the first rotation axis AX1 may be equal to an angle at which the first link is rotated about the first rotation axis AX1.

The user U may seat the tip of his/her finger on the finger seating part 1110 at the same time as holding the handle part 1220, and then, may adduct/abduct the finger about the MCP. In this case, the first link 1120 may be rotated integrally with the finger seating part 1110.

In this manner, the sensor part S may detect the rotation angle of the first link 1120 with respect to the first rotation axis AX1, and the abduction/adduction angle of the MCP may be tracked through the detected rotation angle of the first link 1120.

Specifically, referring to FIG. 8, in case that the user U seats his/her finger on the finger seating part 1110 and then abducts/adducts the MCP of the index finger by d° with respect to an imaginary line IL parallel to the rotation central axis CL of the interface 100 for tracking and controlling a motion, the first link 1120 may be rotated by d° with respect to the imaginary line IL due to the rotation of the finger seating part 1110 and the sensor part S may detect the degree of abduction/adduction of the index finger by detecting the rotation angle of the first link 1120.

The master apparatus 10 may control a position and posture of a front end portion 24 of the end effector 22 by using the detected rotation angle of the first link 1120, and a detailed description thereof is provided below.

Referring to FIGS. 4 to 7, the second link 1130 according to an embodiment of the present disclosure may be rotatably connected to the first link 1120 through the second joint part 1160. Specifically, the second link 1130 may be rotated about the second rotation axis AX2 in a relationship with the first link 1120, and the sensor part S may detect the rotation angle of the second link 1130.

In an embodiment, the second joint part 1160 may be arranged to pass through both the second link 1130 and the first link 1120, and the second link 1130 may be rotated about the second rotation axis AX2 in a relationship with the first link 1120.

In an embodiment, an end portion of the second link 1130 may be rotatably connected to the first link 1120 and may be formed in a shape extending in a direction parallel to the rotation central axis CL of the interface 100 for tracking and controlling a motion.

Referring to FIG. 5, the second link 1130 according to an embodiment of the present disclosure may be formed in a shape which is curved in a preset direction. Due to this, in case that the user U bends his/her finger, the bending moment may be effectively transmitted to the second link 1130, and a detailed description thereof is provided in the description of the third link 1140.

In an embodiment, the second rotation axis AX2 may cross the first rotation axis AX1.

In an embodiment, the second rotation axis AX2 may be arranged perpendicular to the second rotation axis AX2.

In an embodiment, an angle formed by the second rotation axis AX2 and the longitudinal central axis of the handle part 1220 may be 30°or less.

In an embodiment, at least one of the second rotation axis AX2, the third rotation axis AX3, and the fourth rotation axis AX4 may be parallel to the flexion/extension rotation axis of the MCP of the user U who seats his/her finger on the finger seating part 1110 at the same time as holding the handle part 1220.

In an embodiment, at least one of the second rotation axis AX2, the third rotation axis AX3, and the fourth rotation axis AX4 may be parallel to a flexion/extension rotation axis of a proximal interphalangeal joint (PIP) of the user U who seats his/her finger on the finger seating part 1110 at the same time as holding the handle part 1220.

In an embodiment, at least one of the second rotation axis AX2, the third rotation axis AX3, and the fourth rotation axis AX4 may be parallel to a flexion/extension rotation axis of a distal interphalangeal joint (DIP) of the user U who seats his/her finger on the finger seating part 1110 at the same time as holding the handle part 1220.

In an embodiment, the second rotation axis AX2 may be parallel to each of the third rotation axis AX3 and the fourth rotation axis AX4.

Biomechanically, the rotation axes of flexion/extension of the MCP, the PIP, and the DIP are parallel or substantially parallel to each other. Accordingly, in the interface 100 for tracking and controlling a motion, the second link 1130, the third link 1140, and the finger seating part 1110 are pivotally connected in series so as to be rotatable about rotation axes parallel to each other, like a human body model. Thus, in case that the user U seats his/her finger on the finger seating part 1110 and performs a motion, the incongruity of the motion is minimized, and the force of finger bending is efficiently transmitted to the plurality of links 1120, 1130, and 1140 and the plurality of joint parts, thereby enabling accurate finger motion tracking.

Referring to FIGS. 4 to 7, the third link 1140 according to an embodiment of the present disclosure may be rotatably connected to the second link 1130 through the third joint part 1170. Specifically, the third link 1140 may be rotated about the third rotation axis AX3 in a relationship with the second link 1130, and the sensor part S may detect the rotation angle of the third link 1140.

Referring to FIG. 5, the third link 1140 according to an embodiment of the present disclosure may be formed in a shape which is curved in a preset direction. This allows the bending moment to be effectively transmitted to the third link 1140 in case that the user U bends his/her finger.

In an embodiment, the second link 1130 may extend along a curve defined by a preset radius of curvature RC1 with a preset point as a center of curvature CC1, and the third link 1140 may extend along a curve defined by a preset radius of curvature RC2 with a point different from the preset point as a center of curvature CC2.

However, the present disclosure is not limited thereto, and the respective areas of the second link 1130 and the third link 1140 may be formed in a shape in which curves defined by different centers of curvature and different radii of curvature are connected.

In an embodiment, in case the second link 1130 is positioned so that the finger of the user U is seated on the finger seating part 1110 at the same time when the palm of the user U comes into contact with the handle part 1220, the second link 1130 may be formed in a shape of a link which is convex in a lateral direction in which a proximal phalanx of the user U or the handle part 1220 is positioned, and the third link 1140 may be formed as a link which is concave toward a middle phalanx of the user U or the handle part 1220.

Referring to FIG. 5, in case that the second link 1130 and the third link 1140 are arranged along a direction (an-X-axis direction in FIG. 5), the second link 1130 may be formed in a shape which is concave in another direction (Y-axis direction in FIG. 5) and the third link 1140 may be formed in a shape which is convex in the other direction.

Due to this, because the second link 1130 and the third link 1140 are formed in a shape of a curved link, the rotational moment which is transmitted to the second link 1130 and the third link 1140 by the finger motion of the user U is effectively transmitted to the second link 1130 and the third link 1140, and thus, there is an effect in which the sensor part S attached to the second to fourth joint parts 1180 may accurately detect the sum of the bending angles of the MCP, the PIP, and the DIP of the user U.

Hereinafter, a method of detecting the motion of the finger by using the plurality of links 1120, 1130, and 1140 and the joint parts is described.

Referring to FIGS. 4 to 8, each of the second joint part 1160, the third joint part 1170, and the fourth joint part 1180 may be capable of only one-DOF rotation about the second rotation axis AX2, the third rotation axis AX3, and the fourth rotation axis AX4, which are non-parallel to the first rotation axis AX1. Accordingly, the finger seating part 1110, the first link 1120, the second link 1130, the third link 1140, and a fourth link are rotated integrally about the first rotation axis AX1.

Specifically, in case that the user U provides a rotational force based on the first rotation axis AX1 to the finger seating part 1110, the rotational force is not transmitted to the second joint part 1160, the third joint part 1170, and the fourth joint part 1180, and all the rotational force transmitted by the user U toward the first rotation axis AX1 is transmitted to the first joint part 1150.

Referring to FIGS. 6 to 8, the user U may hold the handle part 1220 and then seat the tip of his/her finger on the finger seating part 1110. In this case, in case that the user U adducts or abducts his/her finger by d°, the finger seating part 1110, the first link 1120, the second link 1130, the third link 1140, and the fourth link are rotated integrally about the first rotation axis AX1 by d°, and the sensor part S connected to the first joint part 1150 may detect the rotation angle toward the first rotation axis AX1.

Accordingly, the master apparatus 10 may calculate the degree of adduction/abduction of the finger by using the rotation angle of the first joint part 1150 toward the first rotation axis AX1, which is detected by the sensor part S. In this manner, the master apparatus 10 may control the vector value V1 of the front end portion 24 of the end effector 22 (‘the vector value V1 of the front end portion 24’refers to the position and posture value of the longitudinal central axis of the front end portion).

Referring to FIG. 7, the user U may flex or extend his/her finger. Specifically, the user U may flex or extend the MCP by θ_{1°} , the PIP by θ_{2°} , and the DIP by θ_{3°}. According to the motion of the finger seating part 1110, the second joint part 1160 connected in series to the finger seating part 1110 may be rotated about the second rotation axis AX2 by a°, the third joint part 1170 may be rotated about the third rotation axis AX3 by b°, and the fourth joint part 1180 may be rotated about the fourth rotation axis AX4 by c°.

In this case, under the constraint condition of θ₁+θ₂+θ₃=a+b+c, the interface 100 for tracking and controlling a motion may calculate the bending angle (a+b+c) of the tip of the finger, based on a line parallel to the imaginary line IL, by using an angle (θ₁+θ₂+θ₃) by which the tip of the finger is rotated in a counterclockwise direction (in FIG. 7) based on the imaginary line IL.

In this manner, the master apparatus 10 may control the position and posture of the front end portion 24 of the end effector 22 by using the calculated bending angle of the tip of the finger.

Referring to FIGS. 4 to 6, the finger seating part 1110 according to an embodiment of the present disclosure allows the user U to seat his/her finger thereon and may include a seating body 1111 and a haptic module 1112.

In an embodiment, the seating body 1111 may be formed in a shape of a thimble which allows the tip of the finger to be put thereon or inserted thereinto. However, the present disclosure is not limited thereto, and the seating body 1111 may be formed in various shapes which are capable of covering the tip of the finger.

In an embodiment, the seating body 1111 may be formed of an elastic material, such as rubber. This has an effect of enabling the seating body 1111 to accommodate tips of fingers with various sizes.

The haptic module 1112 according to an embodiment of the present disclosure may be arranged on the inner circumferential surface of the seating body 1111.

In an embodiment, the haptic module 1112 may feed tactile information or a force applied to the end effector 22 during surgery back to the user U.

However, the present disclosure is not limited thereto, and the haptic module 1112 may transmit, to the user U, various forms of sensory information, such as temperature information or pressure information, which the end effector 22 may obtain during surgery.

In an embodiment, the haptic module 1112 may include a pressure sensor or a vibrator capable of applying sensation including a force or a vibration so as to transmit, to a finger, a reaction force applied to a surgical member 23 in case that the surgical member 23 takes a motion, such as holding internal human tissue.

In an embodiment, the haptic module 1112 may be implemented as a tactile display which transmits electrical or mechanical stimulation and may transmit sensation to the finger of the user U. However, the present disclosure is not limited thereto, and the haptic module 1112 may include at least one of various types of pressure, temperature, and friction sensors.

Referring to FIGS. 3, 4, 7, and 8, the base part 1200 according to an embodiment of the present disclosure may include the base frame 1210, the handle part 1220, and the fastening part 1230.

The base frame 1210, which connects the handle part 1220 to the main body part 1100, may extend from a side of the handle part 1220 to an area where the first link 1120 is positioned.

In an embodiment, the base frame 1210 may be rotatably connected to the first link 1120.

The handle part 1220 according to an embodiment of the present disclosure may include a handle body 1221 and a manipulation part 1222.

The handle body 1221 may be formed in various handle shapes to facilitate the holding of the user U.

In an embodiment, an angle formed by the longitudinal central axis of the handle body 1221 and the rotation central axis CL of the base frame 1210 may be 45°or less.

In an embodiment, the longitudinal central axis of the handle body 1221 and the rotation central axis CL of the base frame 1210 may be perpendicular to each other.

In an embodiment, an angle formed by the longitudinal central axis of the handle body 1221 and the first rotation axis AX1 may be 30°or less.

In an embodiment, the longitudinal central axis of the handle body 1221 and the first rotation axis AX1 may be perpendicular to each other.

In an embodiment, the outer circumferential surface of the handle body 1221 may be formed in a curved shape to guide areas where the index finger, middle finger, ring finger, and little finger are respectively seated.

Although not illustrated in the drawings, a detection sensor which may detect whether the user U has held the handle body 1221 may be provided on a side of the handle body 1221.

In an embodiment, the detection sensor may include any one of an optical sensor, a capacitive sensor, and a mechanical sensor.

In an embodiment, the master apparatus 10 may determine whether the user U has held the handle body 1221 through the detection sensor. In case that the master apparatus 10 determines that the user U has not held the handle body 1221, the master apparatus 10 may restrict the driving of the slave robot 20.

In other words, only in case that the detection sensor detects the presence of the hand of the user U, the master apparatus 10 may selectively drive the slave robot 20 by transmitting an electric signal to the slave robot 20, and in case that the detection sensor does not detect the presence of the hand of the user U, the master apparatus 10 does not transmit an electric signal to the slave robot 20 and may restrict the driving of the slave robot 20.

Accordingly, in a situation where the user U does not hold the handle part 1220, the slave robot 20 is prevented from being driven by the operation of the interface 100 for tracking and controlling a motion, which is not intended by the user U, thereby ensuring the safety of surgery.

Referring to FIG. 4, the manipulation part 1222 according to an embodiment of the present disclosure selectively generates a preset control command as the user U manipulates the manipulation part 1222, and may be connected to a side of the handle body 1221.

The control command may include a control signal that selectively drives the surgical member 23 to close or open. However, the present disclosure is not limited thereto, and the control command may include a control signal for controlling various driving methods necessary for the surgical member 23 to perform surgery, such as a control signal for driving the surgical member 23 to emit light, a control signal for driving the surgical member 23 to cut body tissue, etc.

In an embodiment, the manipulation part 1222 may be manipulated by the thumb of the user U, and in case that the user U holds the handle body 1221, the manipulation part 1222 may be positioned in an area of the handle body 1221 corresponding to the position of the thumb.

In an embodiment, the manipulation part 1222 may include input devices having various shapes, such as a button-type switch, a touch sensor, or a joystick.

Referring to FIGS. 3, 4, and 8, the fastening part 1230 according to an embodiment of the present disclosure may be fastened to end portions of robot arm 2100a, 2100b, and 2100c of the manipulator 200 so that the interface 100 for tracking and controlling a motion and the manipulator 200 are integrally operated, and may be connected to a side of the handle part 1220 or the base frame 1210.

In an embodiment, the fastening part 1230 may be rotatably connected to the end portions of the robot arms 2100a, 2100b, and 2100c, and the rotation central axis of the fastening part 1230 may coincide with the rotation central axis CL of the interface 100 for tracking and controlling a motion.

In an embodiment, the fastening part 1230 may be detachably connected to the base frame 1210 or the handle part 1220. Accordingly, because the type of fastening part 1230 connected to the base frame 1210 or the handle part 1220 may be changed depending on the type of manipulator 200 connected to the interface 100 for tracking and controlling a motion, the interface 100 for tracking and controlling a motion may be coupled to various types of manipulator 200 and the compatibility and versatility of the interface 100 for tracking and controlling a motion may be ensured.

Referring to FIG. 2, the manipulator 200 according to an embodiment of the present disclosure may include a plurality of robot arms 2100a, 2100b, and 2100c connected in series to each other, a rotation body 2300, and a support part 2400.

In an embodiment, the manipulator 200 may include a serial-type robot platform. In case that the interface 100 for tracking and controlling a motion is coupled to the manipulator 200 including the serial-type robot platform, the user U may hold the interface 100 for tracking and controlling a motion within a wide movable range and perform surgery.

The manipulator 200 is applied to conventional robot devices or motion estimation devices in various structures, and thus, a detailed description of the internal structure and operation principle of the manipulator 200 is omitted.

Referring to FIG. 1, the surgical robot system 1 according to an embodiment of the present disclosure may include the slave robot 20 which is driven by receiving an electric signal from the master apparatus 10 according to the movement of the master apparatus 10.

The slave robot 20 may include at least one arm 21. The end effector 22 may be installed in the at least one arm 21, and the end effector 22 may come into direct contact with a surgical site and perform a motion in conjunction with the manipulation of the user U on the master apparatus 10.

In an embodiment, the respective robot joints 2200a, 2200b, and 2200c of the manipulator 200, the handle part 1220, and the first to fourth joint parts 1180 may be rotated according to the motion of the hand of the user U in the master apparatus 10, and the respective rotation angles may be detected by the sensor part S. In this manner, the master apparatus 10 may convert the respective rotation angles into electric signals and transmit the electric signals to the slave robot 20.

The slave robot 20, which receives the electric signal from the master apparatus 10, may control the motions of the arm and the end effector 22 by transmitting the electric signal to the arm and the end effector 22.

Referring to FIG. 2, the end effector 22 may include the surgical member 23 which performs surgery in direct contact with the surgical site, the front end portion 24 which controls the position and posture of the surgical member 23, and the rear end portion 25 which is connected to the arm and controls the position and posture of the front end portion.

In an embodiment, the surgical member 23 may include a skin holder, a suction line, a scalpel, scissors, a grasper, a surgical needle, a needle holder, a staple applier, a cutting blade, and the like, but the present disclosure is not particularly limited thereto, and any known tool necessary for surgery may be used.

In general, the surgical member 23 may be broadly classified into a main surgical tool and an auxiliary surgical tool. Here, the term “main surgical tool” may refer to a tool (e.g., a scalpel, a surgical needle, etc.) which performs direct surgical operations, such as incision, suturing, coagulation, and washing on the surgical site, and the term “auxiliary surgical tool” may refer to an surgical tool (e.g., a skin holder, etc.) which assists the motion of the main surgical tool rather than performing direct surgical operations on the surgical site.

In an embodiment, the front end portion 24, which connects the surgical member 23 to the rear end portion 25, may include a multi-joint arm.

In an embodiment, the front end portion 24 and the surgical member 23 may share a longitudinal central axis with each other.

In an embodiment, the front end portion 24 and the rear end portion 25 may be connected to each other by a multi-joint link.

Referring to FIG. 2, the motion of the front end portion 24 may correspond to the motion of the main body part 1100 of the interface 100 for tracking and controlling a motion in the master apparatus 10. Specifically, the front end portion 24 may perform a bending motion with respect to the rear end portion 25 according to the degree of bending of the first to fourth joints of the main body part 1100.

For example, in case that the first joint part 1150 perform adduction or abduction as the finger seated on the finger seating part 1110 performs adduction or abduction, the front end portion 24 performs a yaw motion with respect to the rear end portion 25.

In addition, in case that the second to fourth joint parts 1160 to 1180 are respectively rotated as the finger seated on the finger seating part 1110 performs extension or flexion, the front end portion 24 performs a pitch motion by the sum of the rotation values of the second to fourth joint parts 1160 to 1180 with respect to the rear end portion 25.

In an embodiment, the rear end portion 25, which connects the front end portion 24 to the arm, may receive a driving force from the arm and perform seven-DOF motion.

In an embodiment, the rear end portion 25 may be formed integrally with the arm 21.

Referring to FIG. 2, the motion of the rear end portion 25 may correspond to the motion of the manipulator 200 and the motion of the base part 1200 of the interface 100 for tracking and controlling a motion in the master apparatus 10.

Specifically, in case that the respective robot joints 2200a, 2200b, and 2200c and the rotation body 2300 of the manipulator 200 connected to the interface 100 for tracking and controlling a motion are rotated according to the movement of the hand holding the handle part 1220, the rear end portion 25 may be driven to move in response to the change in the position of the hand of the user U.

In addition, in case that the handle part 1220 is rotated about the rotation central axis CL as the handle part 1220 performs adduction or abduction of the wrist of the hand holding the handle part 1220, the rear end portion 25 may be rotated about the fifth rotation axis AX5.

As a result, the rear end portion 25 may perform a motion corresponding to the movement of metacarpal bones of the user U, and the front end portion 24 may perform a motion corresponding to the movement of a distal phanlax of the user U.

Referring to FIG. 9, a master apparatus 10′ according to another embodiment of the present disclosure has the same configuration, operation principle, and effects as the master apparatus 10′ according to an embodiment of the present disclosure, except for a manipulator 200′ and a base part 1200′, and thus, a detailed description thereof is omitted within an overlapping scope.

The master apparatus 10′ according to another embodiment of the present disclosure may include a manipulator 200′ including a plurality of robot arms 2100a′, 2100b′, and 2100c′ connected in parallel to each other, and the master apparatus 10′ according to another embodiment of the present disclosure may be, for example, a delta robot, which is one of parallel link structure robots.

A base frame 1210′ according to another embodiment of the present disclosure may be formed in a ring shape which is positioned perpendicular to a rotation central axis of the manipulator 200′ for tracking and controlling a motion. However, the present disclosure is not limited thereto, and the base frame 1210′ may be formed in various shapes which may respectively connect a handle part 1220′ to the plurality of robot arms 2100a′, 2100b′, and 2100c′ connected in parallel to each other.

A plurality of fastening parts 1230′according to another embodiment of the present disclosure may be provided to correspond to the number of robot arms 2100a′, 2100b′, and 2100c′ arranged in parallel to each other.

Referring to FIG. 10, the interface 100′for tracking and controlling a motion according to an embodiment of the present disclosure may be directly coupled with a surgical instrument 30 to constitute a laparoscopic surgical apparatus 2.

The surgical instrument 30 may include an end effector 32 for surgery and a shaft 31 which transmits a driving force for the motion of the end effector 32.

Specifically, the end effector 32 may be connected to an end portion of the shaft 31, and a fastening part 1230 of the interface 100 for tracking and controlling a motion may be connected to another end portion of the shaft 31.

In the present specification, the surgical instrument 30 may include various types of surgical instrument 30 which may be inserted into a patient's body to perform surgery, and the shape of the base part 1200 may be modified to correspond to the shape of the surgical instrument 30 connected to the interface 100 for tracking and controlling a motion.

In the laparoscopic surgical apparatus 3 according to an embodiment of the present disclosure, a method of controlling the driving of the end effector 32 by the motion of the interface 100 for tracking and controlling a motion is the same as the method by which the interface 100 for tracking and controlling a motion in the surgical robot system 1 according to an embodiment of the present disclosure controls the driving of the end effector 32, and therefore, a detailed description thereof is omitted within an overlapping range.

Therefore, it will be understood that the spirit of the present disclosure should not be limited to the embodiments described above, and the claims and all equivalent modifications fall within the scope of the present disclosure.

### Industrial Applicability

According to an embodiment of the present disclosure, an interface for tracking and controlling a motion is provided. In addition, embodiments of the present disclosure may be applied to motion tracking and manipulation devices used in industry.

1. An interface for tracking and controlling a motion, the interface comprising:
a base part comprising a handle part which is holdable by a user; and
a main body part comprising a plurality of links pivotally connected to each other and a finger seating part on which a finger of the user is seated, the main body part being rotatably connected to the base part,
wherein at least one of the plurality of links is formed in a shape which is curved in a preset direction.

2. The interface of claim 1, wherein the plurality of links comprise a first link connected to the base part so as to be rotatable about a first rotation axis, a second link connected to the first link so as to be rotatable about a second rotation axis, and a third link connected to the second link so as to be rotatable about a third rotation axis and connected to the finger seating part so as to be rotatable about a fourth rotation axis.

3. The interface of claim 2, wherein the second rotation axis, the third rotation axis, and the fourth rotation axis are parallel to each other.

4. The interface of claim 2, wherein the first rotation axis and the second rotation axis cross each other.

5. The interface of claim 4, wherein the first rotation axis and the second rotation axis are arranged perpendicular to each other.

6. The interface of claim 3, wherein an angle formed by the second rotation axis and a longitudinal central axis of the handle part is 30° or less.

7. The interface of claim 2, wherein the first rotation axis and a longitudinal central axis of the handle part cross each other.

8. The interface of claim 7, wherein the first rotation axis and the longitudinal central axis of the handle part are arranged perpendicular to each other.

9. The interface of claim 2, wherein the main body part comprises:
a first joint part which rotatably connects the base part to the first link;
a second joint part which rotatably connects the first link to the second link;
a third joint part which rotatably connects the second link to the third link; and
a fourth joint part which rotatably connects the third link to the finger seating part,
wherein at least one of the first joint part, the second joint part, the third joint part, and the fourth joint part comprises a sensor part capable of detecting a change in rotation angles of the plurality of links.

10. The interface of claim 9, wherein the sensor part comprises either a magnetic rotary encoder or an optical rotary encoder.

11. The interface of claim 10, wherein a sensor part connected to the first joint part and a sensor part connected to the second joint part comprise different types of encoders.

12. The interface of claim 1, further comprising an imaging part capable of imaging the main body part and the base part,
wherein the imaging part is capable of detecting a position and a rotation angle of at least one of the finger seating part, the plurality of links, and the base part.

13. The interface of claim 9, wherein at least one of the first joint part, the second joint part, the third joint part, and the fourth joint part further comprises a load providing part configured to provide preset torque between links connected to each other.

14. The interface of claim 1, wherein the handle part comprises a manipulation part configured to selectively generate a preset control command according to a manipulation of a user.

15. The interface of claim 1, wherein the finger seating part comprises a haptic module capable of transmitting external environment information to a user.

16. A master apparatus comprising:
a manipulator comprising a plurality of robot joints which are rotatable about one or more non-parallel rotation axes; and
an interface for tracking and controlling a motion which is coupled to an end portion of the manipulator and is capable of tracking a hand motion of a user,
wherein the interface for tracking and controlling a motion comprises:
a base part comprising a handle part capable of coming into contact with a palm of the user and a fastening part coupled to the end portion of the manipulator; and
a main body part comprising a plurality of links pivotally connected to each other and a finger seating part on which a finger of the user is seated, the main body part being rotatably connected to the base part.

17. The master apparatus of claim 16, wherein the plurality of links comprise a first link connected to the base part so as to be rotatable about a first rotation axis, a second link connected to the first link so as to be rotatable about a second rotation axis, and a third link connected to the second link so as to be rotatable about a third rotation axis and connected to the finger seating part so as to be rotatable about a fourth rotation axis.

18. The master apparatus of claim 17, wherein the interface for tracking and controlling a motion is rotatable about a preset axis passing through the fastening part, and the preset axis is spaced apart from the first rotation axis.

19. The master apparatus of claim 16, wherein the fastening part is detachable from the handle part.

20. A laparoscopic surgical apparatus comprising:
a surgical instrument comprising an end effector for surgery and a shaft configured to transmit a driving force for a motion of the end effector; and
an interface for tracking and controlling a motion which is coupled to an end portion of the surgical instrument and is configured to track a hand motion of a user according to a manipulation and transmit hand motion information to the end effector,
wherein the interface for tracking and controlling a motion comprises:
a base part comprising a handle part capable of coming into contact with a palm of the user and a fastening part coupled to the end portion of the surgical instrument; and
a main body part comprising a plurality of links pivotally connected to each other and a finger seating part on which a finger of the user is seated, the main body part being rotatably connected to the base part.

21. A surgical robot system comprising:
a master apparatus comprising a manipulator comprising a plurality of robot joints which are rotatable about one or more non-parallel rotation axes and an interface for tracking and controlling a motion which is coupled to an end portion of the manipulator and is capable of tracking a hand motion of a user; and
a slave robot which is driven by receiving an electric signal from the master apparatus according to a movement of the master apparatus,
wherein the interface for tracking and controlling a motion comprises:
a base part comprising a handle part which is holdable by the user; and
a main body part comprising a plurality of links pivotally connected to each other and a finger seating part on which a finger of the user is seated.
